(19) United States
(12) Patent Application Publication
(10) Pub. No.: US 2012/0294937 A1
(43) Pub. Date: Nov. 22, 2012

(54) NEW PHARMACEUTICAL DOSAGE FORM FOR THE TREATMENT OF GASTRIC ACID-RELATED DISORDERS

(75) Inventor: Andreas Fischer, Uppsala (SE)

(73) Assignee: NOVARTIS AG, Basel (CH)

(21) Appl. No.: 13/519,474

(22) PCT Filed: Dec. 24, 2010
(86) PCT No.: PCT/GB2010/002336

§ 371 Date: Jun. 27, 2012

Related U.S. Application Data
(60) Provisional application No. 61/290,665, filed on Dec. 29, 2009.

(51) Int. Cl. A61K 9/48; A61P 1/04; A61K 31/426; A61K 31/4439; A61K 31/444

(52) U.S. Cl. 424/452; 514/338; 514/303; 514/370

(57) ABSTRACT

According to the invention there is provided a capsule for peroral administration to the gastrointestinal tract containing (a) a pharmacologically effective amount of a PPI or a pharmaceutically acceptable salt thereof and an enteric substance positioned to protect the PPI or salt thereof from the acidic environment of the stomach, and (b) a plurality of granules comprising a pharmacologically effective amount of a micronised H2RA or a pharmaceutically acceptable salt thereof, a disintegrant and a filler The capsules of the invention are particularly useful in the treatment of gastric acid secretion-related disorders, such as gastro-esophageal reflux disease.

This invention relates to new pharmaceutical dosage forms that are useful in the delivery of drugs for the treatment of gastrointestinal disorders.

Gastric acid secretion-related conditions, such as dyspepsia, are common. For example, in the US, around 25% of the adult population experiences heartburn at least weekly. The symptoms of such conditions occur acutely and are at best uncomfortable and at worst extremely painful.

Dyspepsia is a multi-factorial disease and may be associated with organic pathology such as duodenal ulcer, gastric ulcer, esophagitis, Barrett's esophagus or gastro-duodenal inflammation (e.g. Helicobacter Pylori infection). Dyspepsia also includes conditions where no organic pathology can be found, e.g. non-ulcer dyspepsia (NUD) or functional dyspepsia.

Gastro-esophageal reflux disease (GERD) is a related chronic (but often intermittent) disorder typified by abnormal reflux in the esophagus. The disease is characterised by transient or permanent changes in the barrier between the esophagus and the stomach and can arise from a weakening or relaxation of the lower esophageal sphincter, impaired expulsion of gastric reflux from the esophagus or a hiatus hernia. Common symptoms of GERD include heartburn, regurgitation, dysphagia, upper abdominal pain and/or discomfort, excessive salivation and nausea.

It is important that patients obtain immediate relief from such symptoms and for that relief to be sustained for as long as those symptoms continue. Thus, there is a clear unmet clinical need for a rapidly acting, potent and sustained acid-reducing medicament for the symptomatic treatment of gastric acid secretion-related conditions, such as dyspepsia and GERD.

International patent applications WO 02/083132 and WO 2004/035090 disclose the simultaneous co-administration of two classes of anti-secretory agents, proton pump inhibitors (hereinafter “PPIs”) and H2 receptor antagonists (hereinafter “H2RAs”), in the treatment of gastric acid related disorders, such as dyspepsia and GERD.

Simultaneous concomitant therapy, and/or combined dosage forms, comprising these two classes of active ingredients were previously considered counter-intuitive in view of the respective mechanisms of actions of the drugs. In particular, as a PPI's effectiveness was considered to depend upon the degree of activation of acid secretion at the time of drug administration, co-administration with other anti-secretory drugs, such as H2RAs, was never considered appropriate. See, for example, Soil “Peptic Ulcer and Its Complications” in Schlesinger and Fordtran's Gastrointestinal and Liver Disease (Pathophysiology/Diagnosis/Management), 6^{th} Edition (1998) and Wolfe and Sachs “Acid Suppression: Optimizing Therapy for Gastroduodenal Ulcer Healing, Gastroesophageal Reflux Disease, and Stress-Related Erosive Syndrome” Gastroenterology (2000) 118, S9-S31), both of which strongly advise against simultaneous co-administration of PPIs and H2RAs.

In a drug delivery composition comprising H2RAs it is desirable to deliver active ingredient as rapidly as possible in order to provide for immediate relief of symptoms of e.g. GERD.

Granulation (e.g. dry granulation) is a well known process that has hitherto been used to improve powder properties, such as preventing segregation of the ingredients within, improving flow properties of, improving compaction characteristics of, decreasing the bulk volume of, and/or decreasing dust formation of hazardous material within, a powder mix. See, for example, Weyenberg et al, European Journal of Pharmaceutics and Biopharmaceutics, 59, 527 (2005), international patent application WO 97/00682, U.S. Pat. No. 5,576,014 and Canadian patent application No. 2 537 369.

Granulation has thus hitherto been employed to create granulates that act as carriers (see, for example, U.S. Pat. No. 6,261,602 B1) and comprise essentially active substances (see, for example, U.S. Pat. No. 5,622,990). Pharmaceutical compositions comprising a mixture of drug-containing granulates and extra-granular components comprising disintegrants for rapid dissolution are known. See, for example, U.S. Pat. No. 4,609,675, U.S. Pat. No. 6,110,497, U.S. Pat. No. 6,352,720 B1 and U.S. Pat. No. 6,475,501 B1.

According to a first aspect of the invention there is provided a pharmaceutical composition in the form of a capsule for peroral administration to the gastrointestinal tract containing:

(a) a pharmacologically effective amount of a PPI or a pharmaceutically acceptable salt thereof and an enteric substance positioned to protect the PPI or salt thereof from the acidic environment of the stomach; and

(b) a plurality of granules comprising a pharmacologically effective amount of a micronised H2RA or a pharmaceutically acceptable salt thereof, a disintegrant and a filler,

and which compositions are referred to hereinafter as “the compositions of the invention”.

The plurality of granules comprising micronised H2RA or salt thereof, disintegrant and filler is referred to hereinafter as “the granulate”.

Compositions of the invention find particular utility in the field of combination therapies for use in the inhibition of gastric acid secretion. See for example international patent applications WO 02/083132 and WO 2004/035090. As described in those documents, it has been surprisingly found:
(i) firstly, that H2RAs do not compromise the onset of action of acid-susceptible PPIs; and
(ii) secondly, that PPI formulated for delayed and/or extended release may maintain a maximal acid suppression after the first dose and maintain a maximal acid suppression during the course of treatment.

(See also Fändriks et al, Scandinavian Journal of Gasteroenterology (2007) 42, 689.) Prior to the publication of WO 2004/035090 in particular, it was not known that it might be possible to achieve maximal inhibition of acid secretion with an initial PPI dose. However, the latter document describes how expanding the time over which PPI is released in the intestine unexpectedly results in almost complete inhibition of acid secretion as a result of the first dose of PPI. A sustained inhibition of acid secretion may be achieved whilst parietal cells are put into a non-secretory state by means of the rapidly released H2RA.

The present invention is thus especially suitable for “on demand” treatment of gastro-esophageal reflux complaints e.g. heartburn, where potent acid reduction is needed for a reasonably short period of time, that is where a rapid onset of action is important, and maximal acid reduction is preferred. We have found that the maximal acid inhibitory effect may be maintained over a 7 day period in contrast to the “fade-off” phenomenon seen when H2RA is given alone. This is of importance as this aspect of the invention enables a reduction in the time for the treatment of stomach ulcers, acid-related lesions in the esophagus and Helicobacter Pylori eradication.

The term “PPI” will be understood by those skilled in the art to include any compound that is capable of inhibiting gastric H+,K+-ATPase to a measurable degree. Gastric H+,K+-ATPase is the proton-transporting enzyme involved in the production of hydrochloric acid in the stomach. The action of gastric H+,K+-ATPase represents the final step in the sequence of events resulting in secretion of hydrochloric acid by the parietal cell. Thus, inhibition of this enzyme is the most effective and specific means of controlling acid secretion regardless of the nature of the stimulus to secretion. As would be expected with such a mechanism of action, PPIs have been shown to inhibit both basal and stimulated acid secretion.

Particular PPIs that may be utilised in compositions of the invention include acid-susceptible PPIs. The term “acid-susceptible PPI” will be understood by those skilled in the art to include a PPI that acts as a prodrug, in that it accumulates in the acidic milieu of the secretory membrane of the parietal cell before undergoing a chemical transformation in that acid environment to form an active sulphenamide, which irreversibly binds to H+,K+-ATPase by interacting with sulphydryl groups of the acid pump.

As their name suggests, “acid-susceptible PPIs” are generally sensitive to acid and therefore need to be administered in a form which protects them from degradation in the stomach, and to ensure that they are passed into the small intestine where they are absorbed. The term will thus be understood to comprise benzimidazole derivatives, such as omeprazole, pantoprazole, lansoprazole, rabeprazole, pariprazole, tenatoprazole, ilaprazole and leminoprazole, as well as enantiomerically enriched versions of the foregoing, such as dexlansoprazole, estenatoprazole and esomeprazole, and pharmaceutically acceptable salts of any of the foregoing, in addition to compounds disclosed in international patent applications WO 97/25066 (see pages 7 to 11), WO 90/06925, WO 91/19711, WO 91/19712, WO 94/27988 and WO 95/01977, European patent applications EP 005 129 Al, EP 174 726 A1 and EP 166 287 A1, and UK patent application GB 2 163 747, the compounds disclosed generically and specifically in all of which documents are hereby incorporated by reference. Preferred PPIs include esomeprazole, rabeprazole, more preferably dexlansoprazole and particularly lansoprazole or a pharmaceutically acceptable salt thereof.

The term “H2RA” will be understood by those skilled in the art to include any compound that is capable of binding to histamine type 2 (H2 ) receptors, for example those on the surfaces of parietal cells, thereby inhibiting the action of histamine on such receptors and decreasing basal and nocturnal gastric acid secretion, as well as that stimulated by food, insulin and pentagastrin, to a measurable degree.

The term will thus be understood to comprise compounds such as cimetidine, ranitidine, nizatidine, lafutidine, ebrotidine and famotidine, and diastereoisomers and/or enantiomers thereof, and pharmaceutically acceptable salts (e.g. hydrochloride salts) of any of the foregoing. Preferred H2RAs include famotidine or a pharmaceutically acceptable salt thereof.

There are numerous formulation/dosing principles that may be employed in order to prepare compositions of the invention and these are described in a non-limiting sense hereinafter.

However, it is essential that H2RA is, prior to formulation, micronised and thereafter granulated together with a disintegrant and a filler.

By “micronised”, we include that the H2RA is produced in, and/or processed into, a particulate form in which the average particle size diameter is less than about 25 μm, such as less than about 15 μm and preferably less than about 10 μm.

Particle sizes are expressed herein as weight based mean diameters. The term “weight based mean diameter” will be understood by the skilled person to include that the average particle size is characterised and defined from a particle size distribution by weight, i.e. a distribution where the existing fraction (relative amount) in each size class is defined as the weight fraction, as obtained e.g. by sieving.

Primary particles of H2RA may be micronised by techniques that are well known to those skilled in the art, such as grinding, dry milling, jet milling, wet milling, crushing, cutting, precipitation (e.g. by way of dissolution in a supercritical fluid under pressure, followed by rapid expansion) etc, prior to granulation. Primary particles of disintegrant and/or filler may also be processed prior to granulation using similar techniques, although the size of primary particles of disintegrant/filler is not critical.

Disintegrants or disintegrating agents may be defined as materials that are capable of accelerating to a measurable degree the disintegration/dispersion of a component of a composition of the invention, and in particular the granulate. This may be achieved, for example, by the material being capable of swelling and/or expanding when placed in contact with aqueous media (particularly bodily fluids including those found in the gastrointestinal tract), thus causing at least part of a composition of the invention to disintegrate when so wetted. Suitable disintegrants include cross-linked polyvinylpyrrolidone, cross-linked sodium carboxymethylcellulose (croscarmellose, e.g. Ac-Di-Sol, FMC Corp., USA), carboxymethyl starch, natural starch, pre-gelatinised starch, corn starch, potato starch, sodium starch glycolate (Primojel®, DMV International BV, Netherlands), low substituted hydroxypropyl cellulose and the like. Disintegrant (which may comprise one or more of the materials mentioned above) is preferably employed in the granulate in an amount of between about 1% (e.g. about 5%) and about 40% by weight based upon the total weight of the granulate. A preferred range is from about 5% (e.g. about 10%) to about 30% by weight. Preferred disintegrants that are employed in the granulate include cross-linked polyvinylpyrrolidone, cross-linked sodium carboxymethylcellulose, sodium starch glycolate and, particularly, low substituted hydroxypropyl cellulose.

Fillers may be defined as any pharmaceutically acceptable inert material that is capable of increasing the mass of a composition, or a component of a composition, in order to provide an appropriately handleable dosage form. Suitable fillers therefore include (optionally silicified) microcrystalline cellulose, sugars and sugar alcohols (such as lactose, mannitol, xylitol and/or isomalt), calcium phosphate dihydrate and the like. Filler is preferably employed in an amount of between about 5% and about 90% by weight based upon the total weight of the granulate. A preferred range is from about 10% to about 80% by weight. Preferred fillers include mannitol, lactose and xylitol, more preferably, isomalt and microcrystalline cellulose.

Granulate may be prepared by a process of dry granulation, wet granulation, melt granulation, thermoplastic pelletising, spray granulation or extrusion/spheronisation. A preferred technique is dry granulation.

Wet granulation techniques are well known to those skilled in the art and include any technique involving the massing of a mix of dry primary powder particles using a granulating fluid, which fluid comprises a volatile, inert solvent, such as water, ethanol or isopropanol, either alone or in combination, and optionally in the presence of a binder or binding agent. The technique may involve forcing a wet mass through a sieve to produce wet granules which are then dried, preferably to a loss on drying of less than about 3% by weight.

Dry granulation techniques are also well known to those skilled in the art and include any technique in which primary powder particles are aggregated under high pressure, including slugging and roller compaction, for example as described hereinafter.

Melt granulation will be known by those skilled in the art to include any technique in which granules are obtained through the addition of a molten binder, or a solid binder which melts during the process. After granulation, the binder solidifies at room temperature. Thermoplastic pelletising will be known to be similar to melt granulation, but in which plastic properties of the binder are employed. In both processes, the agglomerates (granules) obtained comprise a matrix structure.

Spray granulation will be known by those skilled in the art to include any technique involving the drying of liquids (solutions, suspensions, melts) while simultaneously building up granulates in a fluid bed. The term thus includes processes in which foreign seeds (germs) are provided upon which granulates are built up, as well as those in which inherent seeds (germs) form in the fluid bed due to abrasion and/or fracture, in addition to any spray coating granulation technique generally. The sprayed liquid coats the germs and assists further agglomeration of particles. It is then dried to form granules in the form of a matrix.

Extrusion/spheronisation will be well known to those skilled in the art to include any process involving the dry mixing of ingredients, wet massing along with a binder, extruding, spheronising the extrudate into spheroids of uniform size, and drying.

Granulates comprising H2RA, disintegrant and filler may also comprise other, commonly employed pharmaceutical additives and/or excipients that are used in the art in granulation (see, for example, Pharmaceutical Dosage Forms: Tablets. Volume 1, 2^{nd} Edition, Lieberman et al (eds.), Marcel Dekker, New York and Basel (1989) p. 354-356 and the documents cited therein).

Granulates may thus also comprise other pharmaceutically acceptable excipients known to those skilled in the art, such as binders.

Binders may be defined as materials that are capable of acting as bond formation enhancers, which may facilitate the compression of a powder mass into coherent compacts. Suitable binders include polyvinylpyrrolidone, gelatin, sodium alginate, cellulose derivatives, such as low substituted hydroxypropyl cellulose, hydroxypropyl methylcellulose, cellulose gum, (optionally silicified) microcrystalline cellulose, and the like. If present, binder is preferably employed in an amount of between about 2% and about 50% by weight based upon the total weight of the granulate. A preferred range is from about 5% to about 30% by weight. Preferred binders include cellulose derivatives, such as microcrystalline cellulose, which, as stated above, may also function as a filler, and low substituted hydroxypropyl cellulose, which, as stated above, may also function as a disintegrant.

Granulates may be further processed following formation. For example, a dry granulate may be broken, ground or milled using a suitable milling technique to produce particulate material of a smaller size, which may also be sieved to separate the desired size fraction. Wet granulate may be screened to break up agglomerates of granules and remove fine material. In either case, the unused undersized (fine), and oversized, material may be reworked to avoid waste.

Granulates may be mixed with further additives and/or excipients such as:
(a) lubricants or glidants (such as stearic acid, sodium stearyl fumarate, anhydrous colloidal silica, talc or, preferably, magnesium stearate). When a lubricant is employed it should be used in very small amounts (e.g. up to about 3%, and preferably up to 2%, by weight based upon the total weight of the granulate);
(b) surfactants or wetting agents,

prior to filling into capsules.

The granulate must have a volume small enough to be able to fill in hard shell (size 1, size 2 or size 3) capsules together with PPI, as well as to provide a fast dissolution rate of H2RA in the stomach following administration. In this respect, we prefer that the average granulate size is in the region of about 100 μm to about 2 mm, such as about 200 μm to about 1 mm and, more preferably, about 400 μm to about 800 μm.

H2RA-containing granules of compositions of the invention may be subsequently mixed with a carrier component, in order to ensure that the H2RA and disintegrant (as well as the filler) are distributed homogeneously throughout the composition.

Carrier components may thus be formulated together with the granulate comprising H2RA, disintegrant and filler with a view to keeping granules of the granulate apart (and therefore not agglomerating) within a composition of the invention.

The carrier component may thus comprise one or more pharmaceutically acceptable excipients that are capable of performing such a function. Examples of appropriate materials that may be employed as, or as part of, that carrier component therefore include inert materials that will be well known to those skilled in the art, such as those that are employed in the art as carrier materials, or fillers (vide supra). Suitable materials thus include those mentioned in this context in international patent applications WO 00/16750, WO 2004/06700, WO 2006/103407 and WO 2006/103418, and/or pharmaceutically acceptable inorganic salts, e.g. sodium chloride, calcium phosphate, dicalcium phosphate hydrate, dicalcium phosphate dehydrate, tricalcium phosphate, calcium carbonate, and barium sulfate; polymers, e.g. microcrystalline cellulose, cellulose and crosslinked polyvinylpyrrolidone; starches; sugars and sugar alcohols, e.g. lactose, mannitol, xylitol, isomalt, dextrose; or mixtures of any of the foregoing. Preferred carrier materials include microcrystalline cellulose.

Carrier components if employed may comprise a single material, or often may comprise a combination of materials, some of which may be inert and some of which may perform a specific function. The carrier component may therefore further comprise any of the materials listed hereinbefore as disintegrants, binders, etc.

The carrier component may also comprise further additives and/or excipients, such as:
(a) if it is intended ultimately to form tablets (vide infra), lubricants or glidants (such as stearic acid, sodium stearyl fumarate, anhydrous colloidal silica, talc or, preferably, magnesium stearate). When a lubricant is employed it should be used in very small amounts (e.g. up to about 3%, and preferably up to 2%, by weight based upon the total weight of the composition);
(b) flavourings (e.g. lemon, menthol or peppermint powder), sweeteners (e.g. neohesperidin, sucralose or acesulfame potassium) and dyestuffs;
(c) surfactants;
(d) antioxidants; and/or
(e) other ingredients, such as preservatives and buffering agents.

The carrier component may be admixed with the granulate in accordance with standard mixing techniques. Standard mixing equipment may be used in this regard. Excipients that may collectively make up the carrier component in accordance with the invention may be combined in any order with the granulate.

The mixing time period is likely to vary according to the equipment used, and the skilled person will have no difficulty in determining by routine experimentation a suitable mixing time for a given combination of ingredients. Mixing times are nevertheless selected to ensure that the granulate is homogeneously distributed throughout the carrier. The terms “homogeneous” and “distributed homogeneously” in the context of the present specification mean that there is a substantially uniform content of the granulate component comprising H2RA and intragranular disintegrant throughout the carrier. In other words, if multiple (e.g. at least 30) samples are taken from a mixture of granulate and carrier, the measured content of granulate (and/or H2RA) that is present as between such samples gives rise to a standard deviation from the mean amount (i.e. the coefficient of variation and/or relative standard deviation) of less than about 8%, such as less than about 6%, for example less than about 5%, particularly less than about 4%, e.g. less than about 3% and preferably less than about 2%. If the majority of the granules comprising H2RA are not distributed homogenously within the carrier, the standard deviation from the mean value will be much higher than these values and, as such, this measure is a direct indicator of the “quality” of a composition in terms of potential dose uniformity. The skilled person will appreciate that when such sampling techniques are employed to measure homogeneity, such will take place prior to any compression or compaction step to form e.g. tablets (vide infra).

Granules comprising H2RA may be directly loaded into capsules along with PPI to form compositions of the invention. Alternatively, if granulate is homogenously dispersed within a carrier, such a mixture may be directly loaded into capsules along with PPI, or may be processed into small, discrete units, for example by a process of compression and/or compaction to form, for example, a plurality of pellets or granules, or one or more tablets.

Larger granules comprising granulate according to the invention homogeneously dispersed in carrier component may be made in accordance with techniques described hereinbefore. In such an instance, it is still imperative that granules so formed have a volume small enough to be able to fill in hard shell (size 1, size 2 or size 3) capsules together with PPI, as well as a fast dissolution rate of H2RA in the stomach following administration.

Tablets comprising a homogeneous mixture of granulate in carrier may be formed by a process of compression/compaction. Direct compression/compaction may be achieved using techniques such as those described in, for example, Pharmaceutical Dosage Forms: Tablets. Volume 1, 2^{nd} Edition, Lieberman et al (eds.), Marcel Dekker, New York and Basel (1989) p. 354-356 and the documents cited therein. Suitable compacting equipment includes standard tabletting machines, such as the Kilian SP300 or the Korsch EK0.

Tablets (if employed) need to be of a small size e.g. no more than about 15 mm, such as between about 2 mm and about 12 mm, particularly between about 3 mm and about 10 mm, e.g. between about 3.5 mm and about 6.0 mm, and preferably of a size that enables it to fit into a size 1, a size 2 or a size 3 capsule.

In the PPI-containing component (a) of a composition of the invention, PPI/salt thereof may be presented together with the enteric substance as a powder, or, more preferably, compacted either in pelletised form, i.e. as multiple units (pellets or granules) comprising individual cores of PPI/salt thereof, or as a single unitary central core.

In the context of the present invention, the “enteric” substance is employed and arranged within the PPI-containing component (a) in compositions of the invention such that it is capable of substantially preventing the PPI or salt thereof within that component from being released, and/or coming into contact with gastric juices, until that component reaches the small intestine. By “substantially preventing” we include that no more than about 20%, such as about 15%, for example about 10%, or more particularly no more than about 5%, of PPI/salt is released within the acid environment of the stomach.

Typical enteric coating materials include the following: cellulose acetate, cellulose acetate succinate, cellulose acetate phthalate, cellulose acetate tetrahydrophthalate, polyvinyl acetate phthalate, hydroxyethyl ethyl cellulose phthalate, methacrylic acid copolymers, polymethacrylic acid/acrylic acid copolymers, styrol maleic acid copolymers, hydroxypropyl methyl cellulose phthalate, acrylic resins, cellulose acetate trimellitate, hydroxypropyl methylcellulose trimellitate, shellac, hydroxyethyl ethyl cellulose phthalate, carboxymethylcellulose and hydroxypropyl methyl cellulose acetate succinate. Preferred enteric substances include methacrylic acid copolymers.

Although it is not excluded that the enteric substance may be included within (e.g. admixed with) the PPI and any other excipients that may be present to form a PPI-containing component (a) of a composition of the invention (in the form of a matrix), we prefer that the enteric substance is presented as a discrete coating on the exterior of one or more units comprising PPI or salt thereof (and any other excipients that may be present).

PPI and/or salt thereof is thus preferably provided in the form of one or more units or cores, which units or cores may be in the form of a matrix (i.e. admixed with the enteric substance) as described above, and/or be over-coated with the enteric substance as described above.

PPI/salt in the form of a powder may thus be mixed with excipients, such as fillers, carriers, lubricants etc. (as described above) and processed into units, such as granules, pellets, etc., for example by granulation techniques such as those described hereinbefore, compression and/or by extrusion/spheronisation. Alternatively, pellets and/or granules comprising PPI (and optional excipients) may be blended with further excipients and thereafter compacted into one or more cores, for example as described hereinbefore.

It is preferred that the PPI-containing component of the compositions of the invention is provided in the form of a plurality of (i.e. multiple) units, such as enterically coated pellets, microgranules, etc. In this respect, in a composition of the invention, PPI/salt thereof is preferably presented as multiple units (pellets or granules) comprising individual cores of PPI/salt thereof, which is thereafter mixed with the enteric substance, or individually coated with, or surrounded by, the enteric substance.

Core(s) or multiple units comprising PPI/salt thereof may be covered with a separating layer prior to mixing with, or application of, the enteric substance. The separating layer may serve to provide a moisture barrier and/or a barrier to protect acid susceptible PPI/salt from chemical decomposition brought on by the enteric coatings, which may comprise acidic components. Such a barrier may comprise film-forming agents, such as a sugar, a sugar alcohol, polyethylene glycol, polyvinylpyrrolidone, polyvinyl alcohol, hydroxypropyl cellulose, hydroxymethyl cellulose and/or hydroxypropyl methylcellulose, prior to coating with enteric substance. A preferred material is polyvinyl alcohol (part hydrolysed).

The separating layer may by applied using various techniques, such as a spray-coating technique. In this regard, a film-forming agent, such as one of those described above is applied by pre-dissolving or pre-dispersing it in a solvent, for example an organic solvent, such as acetone, methanol, ethanol, isopropyl alcohol, ethyl acetate and/or methylene chloride or, preferably, an aqueous solvent, such as purified water, followed by spraying or the use of a rotating pan and/or a fluid-bed spray coater. If necessary, pH may be controlled by the polymer or combination of polymers selected and/or ratio of pendant groups in order to control dissolution. Plasticisers, such as triacetin, dibutyl phthalate, polyethylene glycols (e.g. macrogols), triethyl citrate, etc may be included in the spray-coating solution, as well as wetting agents (i.e. surfactants), including polysorbates, sodium lauryl sulphate, lecithin and/or bile acid salts, and glidants and/or lubricants (e.g. talc).

Multiple units comprising PPI and enteric substance are commercially available. Techniques for making them are also described in inter alia U.S. Pat. No. 5,817,338, U.S. Pat. No. 6,328,994 B2, U.S. Pat. No. 7,431,942 B2, 2006/0018964 A1 and Chem. Pharm. Bull., 51, 1121 (2003), the relevant disclosures in which documents are hereby incorporate by reference.

The PPI-containing unit(s) of compositions of the invention may also comprise one or more further excipient materials to provide for a delayed and/or, preferably, an extended release of PPI or salt thereof in the intestines.

The term “extended release” is intended to be synonymous with “prolonged release” and/or “sustained release”, whereby the rate of release of active ingredient is altered, i.e. at a sufficiently retarded rate to produce a therapeutic response over a required period of time. The term “delayed” release is intended to mean the delay of release of active substance for a pre-determined time within the gastrointestinal tract. A substance that provides for a delayed release does not necessarily also provide for an extended release. The essential enteric substance that is included within a composition of the invention provides for enteric release, which is in itself a form of delayed release in that the active substance (PPI) is not released for absorption in the stomach, but rather release is delayed until PPI reaches the small intestine.

The excipient that provides for a delayed and/or extended release of PPI or salt thereof may be associated with PPI-containing units of the compositions of the invention and may therefore be e.g. admixed with the enteric substance or may comprise a separate, discrete coating. Further, it may be applied to PPI in the form of a membrane or may be admixed together with the PPI to form a matrix, in the same way as described hereinafter for the enteric substance.

Such materials, which are well known to those skilled in the art, and may be inert and/or lipid-based. The excipient(s) may therefore comprise non-polymeric or polymeric materials, such as calcium phosphate, ethyl cellulose, methyl cellulose, methacrylate copolymer, hydroxypropyl methylcellulose (hypromellose), polyamide, polyethylene, polyvinyl alcohol or polyvinyl acetate. Lipid-based excipient(s) may comprise non-polymeric or polymeric materials based on fats, such as carnauba wax, cetyl alcohol, hydrogenated vegetable oils, microcrystalline waxes, mono-, di- and triglycerides, polyethylene glycol or polyethylene glycol monostearate. Hydrophilic, pore-forming excipients, such as alginates, carbopol, gelatin, hydroxypropyl cellulose or hydroxypropyl methylcellulose, may also be added.

The enteric substance or enteric substance-containing mixtures, may be applied to the surface(s) of the PPI/salt thereof (in the form of a pellets/multiple units or central cores) using techniques that will be well known to those skilled in the art.

Enteric substance may thus be applied by way of a processing step that comprises press-coating, which will be understood by the skilled person to involve any technique in which a dry powder is compressed in the substantial absence of solvent (although a lubricant may be employed to assist the compaction process) onto another substance (optionally in the presence of other ingredients) using suitable compacting equipment. Appropriate equipment includes standard tabletting machines, such as the Kilian SP300, the Korsch EK0 or the Manesty DryCota Model 900 core and coating tablet press. See, for example, Clausen et al, J. Control. Release (2001) 75, 93 and Schiermeier and Schmidt, Eur. J. Pharm. Sci. (2002) 15, 295.

However, the enteric substance may also be applied by pre-dissolving or pre-dispersing it in a solvent, followed by spraying, application as a chemical vapour, or the use of a rotating pan or a fluid-bed spray coater, using the same techniques as described hereinbefore. In addition to plasticisers, wetting agents and glidants that may be employed in spray-coating solutions that may be employed to make the PPI-containing unit(s) of compositions of the invention, such solutions may further comprise buffering agents, such as sodium bicarbonate or other alkaline-reacting substances, including those described below.

PPI/salt thereof may also be blended with such basic/alkaline-reacting substances, including those described hereinafter, prior to application of the enteric substance, in order to neutralise the small amounts of protons that may be released from the enteric substance during storage and/or may pass through the enteric substance during passage through the stomach.

We have also found unexpectedly that placing H2RA in direct contact with the enteric substance may have a deleterious effect on that substance, reducing its chemical stability and physical integrity. Although preparing H2RA in the form of granules decreases the degree of contact between the H2RA and the enteric substance (when compared to a H2RA in the form of powder), a physical and/or chemical barrier may also be located between the PPI-containing component (a) and the H2RA-containing component (b) of the invention.

A chemical barrier may comprise an acid, such as a fruit acid (e.g. glycolic acid, lactic acid, mandelic acid or, preferably, citric acid).

A physical barrier may comprise a sugar, a sugar alcohol, or a polymer substance, such as a polymer coating, which may comprise e.g. polyethylene glycol, polyvinylpyrrolidone, hydroxypropyl cellulose, hydroxymethyl cellulose, hydroxypropyl methylcellulose or, preferably, polyvinyl alcohol (e.g. part-hydrolyzed polyvinyl alcohol). The physical barrier may be located adjacent to (e.g. on the periphery of and/or surrounding) the PPI-containing unit(s) (a) in a composition of the invention. Such a physical barrier may thus be applied to the PPI-containing units as a coating using techniques such as those described hereinbefore.

PPI-containing component (a), preferably in the form of multiple units as described hereinbefore is packaged and presented together H2RA-containing component (b) of the invention, preferably in the form of granules, in, for example, a suitable, e.g. hard gelatin or hydroxypropyl methylcellulose (size 1, size 2 or size 3), capsule as a single unit dosage form.

Compositions of the invention may further comprise, or be co-administered with, a gastric acid-suppressing agent and/or an alginate. If employed in the composition, 100 mg to 1000 mg of antacid agent and/or alginate may be added. The antacid agent may comprise aluminum hydroxide, calcium carbonate, magnesium carbonate, basic magnesium carbonate, magnesium hydroxide, magnesium oxide and sodium hydrogen carbonate.

Suitable final capsule weights are in the range about 10 mg to about 2 g, such as about 50 mg to about 600 mg. Suitable capsule diameters are in the range about 3 mm to about 20 mm, such as about 4 mm to about 10 mm.

PPIs and H2RAs are employed in pharmacologically effective amounts in compositions of the invention. The term “pharmacologically effective amount” refers to an amount of active ingredient, which is capable of conferring the desired therapeutic effect on a treated patient, depending upon the drug that is employed, whether administered alone or in combination with another active ingredient. Such an effect may be objective (i.e. measurable by some test or marker) or subjective (i.e. the subject gives an indication of, or feels, an effect).

Thus, amounts of PPI and/or H2RA that may be employed in compositions of the invention may be determined routinely by the physician or the skilled person, in relation to what will be most suitable for an individual patient. This is likely to vary with the mode of administration, the nature and severity of the condition that is to be treated, as well as the age, weight, sex, renal function, hepatic function and response of the particular patient to be treated.

However, it is preferred that an H2RA is provided in a composition of the invention in an amount that is effective to reduce acidity in the stomach after administration. It is further preferred that an (acid-susceptible) PPI is provided in such a composition of the invention in an amount that is effective to sustain the reduced acidity effected by the H2RA over an extended period of time. In this respect, it is preferred that the respective amounts are those which are capable of raising gastric pH to a value of above about 3 (preferably above about 4) within about 2 hours of administration, in addition to maintaining this pH level for at least about 4 hours, preferably for at least about 8 hours, more preferably for at least about 16 hours.

Thus, the H2RA may be provided in an amount which is capable of providing at least about 80% (e.g. about 95%) of maximal reduction of the acidity in the stomach within about 2 hours. The term “maximal reduction” will be understood by the skilled person to include the reduction of acidity that can be obtained as a maximum when an equivalent H2RA is administered alone in an equivalent dose in a therapeutically acceptable amount (i.e. an amounts that are accepted dosages in the prior art). Thus, a composition of the invention may comprise between about 1 mg and about 1,000 mg of H2RA or salt thereof, more preferably between about 5 mg and about 400 mg. Preferred dosages for cimetidine are between about 250 mg and about 900 mg; preferred dosages for ranitidine are between about 100 mg and about 400 mg; preferred dosages for famotidine are between about 5 mg and about 50 mg; and preferred dosages for nizatidine are between about 50 mg and about 400 mg.

Accordingly, the PPI may be provided in an amount which is capable of maintaining the low acidity effected by the H2RA over at least about 6 hours. Thus, a composition of the invention may comprise between about 1 mg and about 100 mg, more preferably between about 5 mg and about 75 mg, per single dose of PPI or salt thereof. Preferred dosages for omeprazole and tenatoprazole are between about 5 mg and about 30 mg; preferred dosages for lansoprazole are between about 10 mg and about 40 mg; preferred dosages for pantoprazole are between about 20 mg and about 50 mg; and preferred dosages for esomeprazole are between about 10 mg and about 50 mg; and preferred dosages for dexlansoprazole are between about 20 mg and about 70 mg.

Compositions of the invention are preferably administered by way of a dosing regimen that is capable of maintaining gastric pH above about 3 (e.g. about 4, such as about 5) for at least about 95% of the time, from about 2 hours after administration of the first dose until about 6 hours after the administration of the last dose. Particularly preferred dosing regimens include those in which the dosing period is at least about 1 day (e.g. use on an “as required” basis), e.g. at least about 1 week, preferably about 2 weeks.

The above-mentioned dosages are exemplary of the average case; there can, of course, be individual instances where higher or lower dosage ranges are merited, and such are within the scope of this invention.

Compositions of the invention may be administered once or several times a day, for example perorally by way of appropriate dosing means known to the skilled person. In this respect, the compositions of the invention may be incorporated into various kinds of pharmaceutical preparations intended for oral administration using standard techniques (see, for example, Lachman et al, “The Theory and Practice of Industrial Pharmacy”, Lea & Febiger, 3^{rd} edition (1986) and “Remington: The Science and Practice of Pharmacy”, Gennaro (ed.), Philadelphia College of Pharmacy & Sciences, 19^{th} edition (1995)).

Compositions of the invention may be employed to provide both rapid onset of inhibition of gastric acid secretion, followed by maintenance of such inhibition as long as desired (for example by repeated administration of PPI preferably in the form of a composition of the invention). Thus, compositions of the invention are useful in the (e.g. symptomatic) treatment of dyspepsia and other gastrointestinal disorders related to the production of gastric acid, such as dyspepsia, GERD, etc.

Compositions of the invention may also be useful in a treatment program designed for the healing of gastric and duodenal ulcers, and esophagitis, for which the maintenance of intragastric pH above 4 for a maximal duration should be attained (see Huang J Q and Hunt R H, pH, Healing Rate and Symptom Relief in Patients with GERD, Yale J Biol Med 1999, 72:181-94).

Compositions of the invention may also be used, in association with one or more antibiotic agent(s), for the eradication of Helicobacter pylori.

According to a further aspect of the invention, there is provided a method of treatment of a disorder associated with gastric acid secretion, such as dyspepsia, GERD, gastric ulcers, duodenal ulcers, oesophagitis, Barrett's oesophagus, oesophageal adenoma, gastric cancer and the like, which method comprises administration of a composition of the invention to a patient in need of such treatment.

Compositions of the invention are particularly useful in the treatment (such as the “on demand” treatment) of GERD, and in particular treatment of the symptoms thereof, including heartburn, regurgitation, indigestion, dysphagia, upper abdominal pain and/or discomfort, excessive salivation, sour stomach and nausea.

For the avoidance of doubt, by “treatment” we include the therapeutic treatment, as well as the symptomatic treatment, the prophylaxis, or the diagnosis, of a condition.

Wherever the word “about” is employed herein in the context of dimensions (e.g. sizes, weights, pH values, time intervals, etc.), amounts (e.g. relative amounts of individual constituents in a composition or a component of a composition, absolute doses of active ingredient, degrees of release of active ingredients, reductions in gastric acidity, standard deviations, and other percentages), it will be appreciated that such variables are approximate and as such may vary by ±10%, for example ±5% and preferably ±2% (e.g. ±1%) from the numbers specified herein.

The compositions of the invention are easy and inexpensive to manufacture, and enable the rapid and sustained relief of the symptoms described hereinbefore.

Compositions of the invention have surprisingly been found to exhibit a rapid rate of dissolution of H2RA at high pHs. Firstly, this means that compositions of invention exhibit a rapid dissolution of H2RA that is independent of pH. Rapid dissolution (and therefore availability for absorption) may therefore take place over a wider region of the gastrointestinal tract (e.g. both the stomach and in the smaller intestine). Secondly, this means that compositions of invention exhibit a rapid dissolution of H2RA that is not compromised in patients exhibiting high pH values in the stomach, for example because they are receiving gastric acid suppression therapy (and particularly a more effective therapy such as one comprising a combination of H2RA and PPI, as described hereinbefore).

Compositions of the invention may also have the advantage that they may be prepared using established pharmaceutical processing methods and employ materials that are approved for use in foods or pharmaceuticals or of like regulatory status.

Compositions of the invention may also have the advantage that they may be more efficacious than, be less toxic than, be longer acting than, be more potent than, produce fewer side effects than, be more easily absorbed than, and/or have a better pharmacokinetic profile than, and/or have other useful pharmacological, physical, or chemical properties over, pharmaceutical compositions known in the prior art, whether for use in the treatment of gastrointestinal disorders related to the production of gastric acid (e.g. dyspepsia and GERD), the eradication of Helicobacter pylori, or otherwise.

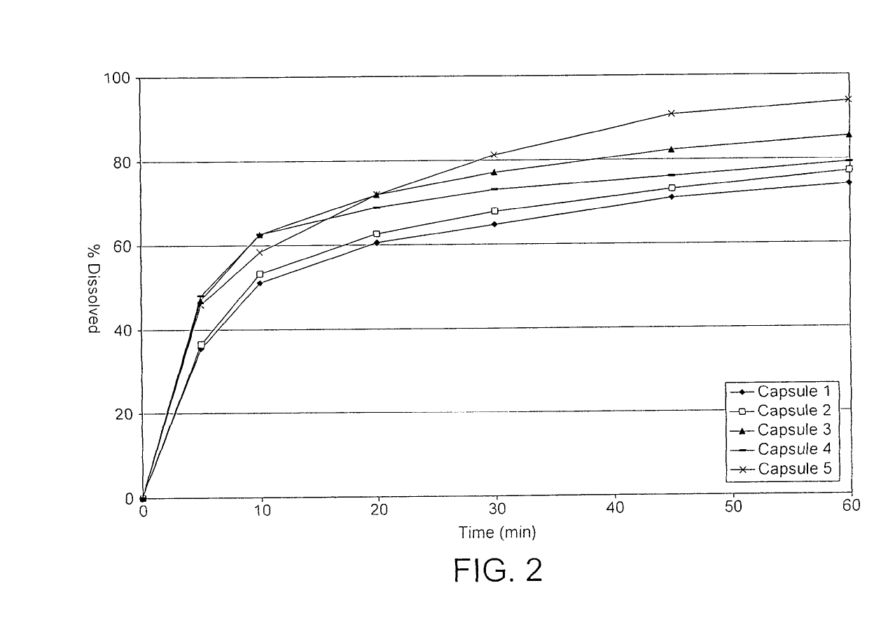

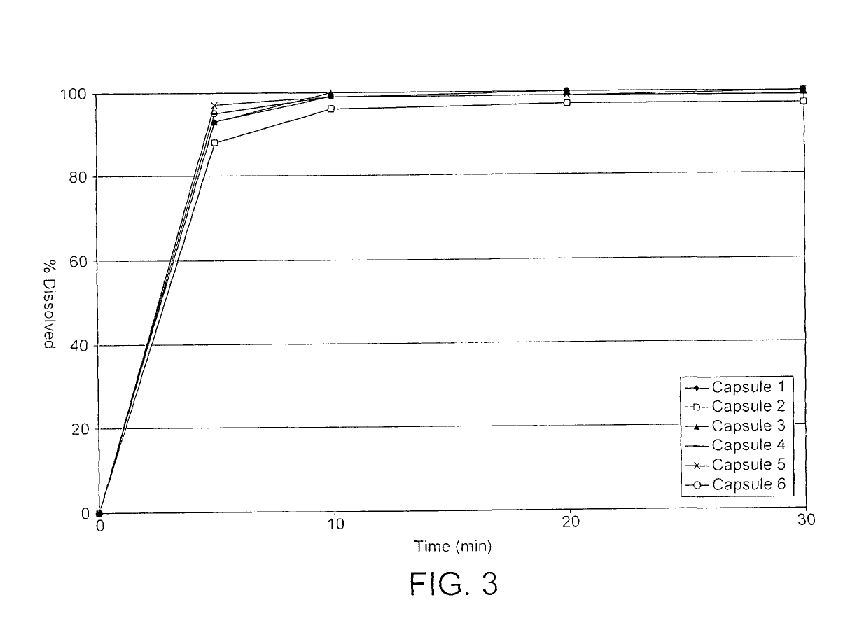

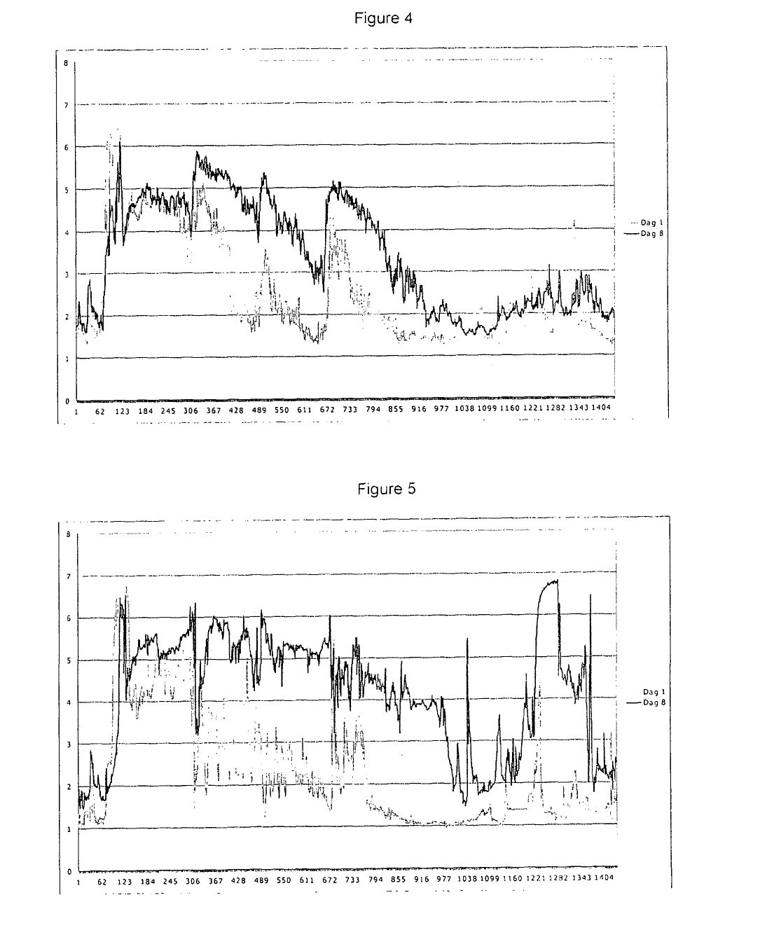

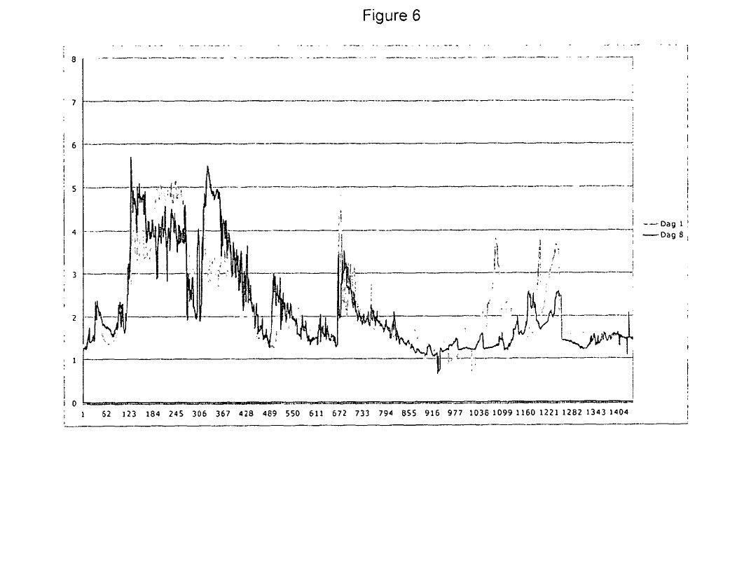

The invention is illustrated by way of the following examples in which FIGS. 1 to 3 show the release of famotidine at pH 7 from granules made in accordance with the invention (FIGS. 1 and 3), and granules not made in accordance with the invention (FIG. 2), loaded into capsules; and FIGS. 4 to 6 show pH profiles (y-axis) over several days (hours represented on x-axis) as a consequence of concomittant co-administration of H2RA and PPIs (omprazole, esomeprazole and lansoprazole, respectively).

### Example 1

### Famotidine Granules and Lansoprazole Pellets in Capsule

Famotidine (Gedeon Richter; weighed out in an amount provide 10 mg per capsule), isomalt (Beneo-Palatini; weighed out in an amount provide 10 mg per capsule), microcrystalline cellulose (FMC BioPolymer; weighed out in an amount provide 5 mg per capsule), crospovidone (International Specialty Products; weighed out in an amount provide 5 mg per capsule) and croscarmellose (FMC BioPolymer; weighed out in an amount provide 10 mg per capsule) were mixed together (Turbula mixer, 47 rpm) for 70 minutes. The resultant mixture was dry granulated together by a process of slugging (Korsch EK0, circular flat tableting tools, 20 mm in diameter) followed by screen milling (Erweka 200AR) through a 2.50 mm mesh and finally a 1.00 mm mesh.

The resultant famotidine containing granules were sieved on a 300 μm mesh and the undersize fraction (below 300 μm) was once more dry granulated, screen milled and sieved as above. The two famotidine containing fractions between about 300 μm and about 1000 μm were pooled into one final famotidine granulate. The final granulate had a volume weighted mean diameter (d (0.5)) of about 695 μm (measured by laser diffraction using a Malvern MasterSizer 2000).

Enterically-coated lansoprazole capsule pellets (8% API extracted from commercially available delayed release lansoprazole capsules (Prevacid® 15 mg; Novartis)) were weighed out in an amount to provide 188 mg (15 mg API) weight per final capsule and loaded, along with famotidine-containing granulate (prepared as described above) into hard shell gelatin size 2 capsules.

Five capsules were tested for dissolution according to USP <711> Apparatus 2 (Paddle Apparatus), with sinker baskets, at 50 rpm and 37° C. in 900 mL of USP pH 7.0 phosphate buffer solution. Samples were withdrawn at 5, 10, 20, 30, 45 and 60 minutes. The aliquots withdrawn for analysis were not replaced; the volume change was corrected for in the calculation. The analysis were performed by HPLC according to USP <621> High-Pressure Liquid Chromatography, with a L1 column. The results are shown in FIG. 1.

### Example 2 (Comparative)

### Famotidine Granules and Lansoprazole Pellets in Capsule

Famotidine (Gedeon Richter; weighed out in an amount provide 10 mg per capsule), isomalt (Beneo-Palatinit; weighed out in an amount provide 20 mg per capsule), microcrystalline cellulose (FMC BioPolymer; weighed out in an amount provide 10 mg per capsule) were mixed together (Turbula mixer, 47 rpm) for 70 minutes. The resultant mixture was dry granulated and further processed as described in Example 1. The final granulate had a volume weighted mean diameter (d(0.5)) of about 639 μm (measured by laser diffraction using a Malvern MasterSizer 2000).

The granulate was loaded with equivalent enterically-coated lansoprazole capsule pellets (as in Example 1) into hard shell gelatin size 2 capsules and tested dissolution as described in Example 1. The results are shown in FIG. 2.

It can be seen that the dissolution rate at pH 7.0 is much faster for the composition of Example 1.

### Example 3

### Famotidine Granules and Lansoprazole Microgranules in Capsule

Famotidine (Gedeon Richter; weighed out in an amount provide 10 mg per capsule), isomalt (Beneo-Palatini; weighed out in an amount provide 27 mg per capsule), microcrystalline cellulose (FMC BioPolymer; weighed out in an amount provide 70.73 mg per capsule) and low substituted hydroxypropyl cellulose (Shin-Etsu Chemical Co.; weighed out in an amount provide 27 mg per capsule) were mixed together (Turbula mixer, 25 rpm) for 15 minutes. The resultant mixture was then dry granulated by roller compaction and sieving in a dry granulator (W120 Pharma, Alexanderwerk GmbH, Germany) using roller speed of 5 rpm, roller pressure of 15 kN/cm, wafer thickness of 2 mm, screen mill impeller speed 25 rpm and pore sizes of primary and secondary screens of 3.15 mm and 1.25 mm respectively.

The resultant famotidine containing granules (first fraction) were sieved on a 315 μm mesh and the undersize fraction (below 315 μm) was once more dry granulated, screen milled and sieved as above (second fraction). The second fraction was also reworked as above to produce a third fraction. The three famotidine containing fractions between about 315 μm and about 1250 μm were pooled into one final famotidine granulate.

Magnesium stearate (Peter Greven; weighed out in an amount to provide 0.27 mg per capsule) was then added to the granulate and mixed together (Turbula mixer, 25 rpm) for 5 minutes.

Enterically-coated lansoprazole microgranules (11.1% API, as described in Chem. Pharm. Bull., 51, 1121 (2003)) were weighed out in an amount to provide 135 mg (15 mg API) weight per final capsule and loaded, along with famotidine-containing granulate (prepared as described above) into hard shell gelatin size 2 capsules (Qualicaps) using a capsule filling machine (Bosch GKF 400, Bosch, Germany).

Six capsules were tested for dissolution according to USP <711> Apparatus 2 (Paddle Apparatus), with sinker baskets, at 50 rpm and 37° C. in 900 mL of USP pH 7.0 phosphate buffer solution. Samples were withdrawn at 5, 10, 20 and 30 minutes. The aliquots withdrawn for analysis were not replaced; the volume change was corrected for in the calculation. The analysis were performed by HPLC according to USP <621> High-Pressure Liquid Chromatography, with a L1 column. The results are shown in FIG. 3.

### Example 4

### Famotidine Granules and Lansoprazole Microgranules in Capsule

Famotidine (Gedeon Richter; weighed out in an amount provide 10 mg per capsule), isomalt (Beneo-Palatini; weighed out in an amount provide 27 mg per capsule), microcrystalline cellulose (FMC BioPolymer; weighed out in an amount provide 70.73 mg per capsule) and low substituted hydroxypropyl cellulose (Shin-Etsu Chemical Co.; weighed out in an amount provide 27 mg per capsule) were mixed together (Turbula mixer, 25 rpm) for 25 minutes. The resultant mixture was then dry granulated by roller compaction and sieving in a dry granulator (W120 Pharma, Alexanderwerk GmbH, Germany) using roller speed of 5 rpm, roller pressure of 15 kN/cm, wafer thickness of 2 mm, screen mill impeller speed 25 rpm and pore sizes of primary and secondary screens of 3.15 mm and 1.25 mm respectively.

The resultant famotidine containing granules were sieved on a 315 μm mesh and the oversize fraction (between about 315 μm and about 1250 μm) was collected as the final famotidine granulate.

Magnesium stearate (Peter Greven; weighed out in an amount to provide 0.27 mg per capsule) was then added to the granulate and mixed together (Turbula mixer, 46 rpm) for 2 minutes.

Enterically-coated lansoprazole capsule pellets (Sandoz; 7.8% API) were weighed out in an amount to provide 193 mg (15 mg API) weight per final capsule and loaded, along with famotidine-containing granulate (prepared as described above) into hard shell gelatin size 1 capsules.

The capsules were placed in high density polyethylene bottles without desiccant and stored at 40° C. and 75% relative humidity. After 6 months 10 capsules were tested for organic impurities of lansoprazole. The analysis was performed by HPLC according to USP <621> High-Pressure Liquid Chromatography, with a L1 column. The total amount of organic impurities was below 0.1 area-%.

### Example 5 (Comparative)

### Famotidine Powder Mixture and Omeprazole Pellets in Capsule

Famotidine (Quimico Sintetica; weighed out in an amount provide 10 mg per capsule), mannitol (Roquette; weighed out in an amount provide 103 mg per capsule) were mixed together (Turbula mixer, 72 rpm) for 24 hours. The resultant mixture was the famotidine powder mixture.

Enterically-coated omeprazole capsule pellets (Union Quimico Farmaceutica; 8.6% API) were weighed out in an amount to provide 233 mg (20 mg API) weight per final capsule and loaded, along with famotidine powder mixture (prepared as described above) into hard shell HPMC size 0 capsules.

The capsules were placed in aluminum sachets without desiccant and stored at 40° C. and 75% relative humidity. After 6 months 5 capsules were tested for organic impurities of omeprazole. The analysis was performed by HPLC according to USP <621> High-Pressure Liquid Chromatography, with a L1 column. The total amount of organic impurities was 13.4 area-%.

Conclusions from Examples 4 and 5

The higher total organic impurities of the PPI omeprazole in Example 5, compared to the PPI lansoprazole in Example 4 demonstrates the improved chemical stability of the PPI when the H2RA is prepared in the form of granules, compared to H2RA prepared in the form of powder.

Clinical Data

In support of the efficacy of the claimed invention, reference is made to Fandriks et al, Scandinavian Journal of Gasteroenterology (2007) 42, 689-694, the clinical protocols and data presented in which are hereby incorporated by reference, which demonstrate, in the clinical setting, that famotidine (H2RA) and omeprazole (PPI) may be co-administered, and actually provide an additive benefit in the early days of treatment.

The data compare the control arms omeprazole alone; famotidine alone; and co-administration of omeprazole and famotidine. The data show for Day 1 (i) omeprazole alone controlled stomach acid pH>4 for 27% of the day, (ii) famotidine alone controlled stomach acid pH>4 for 54% of the day; and (iii) the co-administration of omeprazole and famotidine controlled the stomach acid pH>4 for 67% of the day.

A surprising effect is therefore observed on Day 1, given the traditional view that an adverse effect would be observed due to the co-administration, because of the contemporaneous co-administration of a PPI and an H2RA.

On Day 8, when the steady state for the omeprazole had been reached, the percentage of time that omeprazole alone was controlling stomach acid was 78% of the day (which is in keeping with the pharmacological profile of a PPI); famotidine alone was controlling stomach acid for 48% of the day, which is similar to the 54% on Day 1, but showing evidence of physiological tolerance (also in keeping with the pharmacological profile of a H2RA). The co-administration of PPI and H2RA was controlling stomach acid for 78% of the day, showing none of the expected diminished PPI activity compared to Day 1.

According to the prior conventional wisdom, on Day 1 and Day 8 the control percentages for the co-administered dose would be expected to be lower than the individual doses. At the very least, the co-administered dose would be expected to be lower on Day 8 compared to the omeprazole alone, because the effectiveness of omeprazole would be expected to be inhibited by the presence of H2RA.

Subsequent studies have demonstrated that the co-administration of other PPIs with famotidine provide similar surprising results. Studies of the PPI esomeprazole co-administered with famotidine and of the PPI lansoprazole co-administered with famotidine, each pair being administrated simultaneously or concomitantly on a once daily basis for eight days, demonstrate that the co-administration of these PPIs with an H2RA simultaneously or concomitantly gives an early and clinically important intragastric pH increase within two hours after co-administration of the first dose, and that the acid-suppressive effect is maintained throughout the eight days treatment period. The results are shown in FIGS. 4 to 6, respectively.

1. A capsule for peroral administration to the gastrointestinal tract containing:
(a) a pharmacologically effective amount of a PPI or a pharmaceutically acceptable salt thereof and an enteric substance positioned to protect the PPI or salt thereof from the acidic environment of the stomach; and
(b) a plurality of granules comprising a pharmacologically effective amount of a micronised H2RA or a pharmaceutically acceptable salt thereof, a disintegrant and a filler.

2. A capsule as claimed in claim 1, wherein the proton pump inhibitor is omeprazole, pantoprazole, lansoprazole, rabeprazole, pariprazole, tenatoprazole, ilaprazole or leminoprazole, or an enantiomer thereof, or a pharmaceutically acceptable salt of any of the foregoing.

3. A capsule as claimed in claim 2, wherein the enantiomer is estenatoprazole, dexlansoprazole or esomeprazole, or a pharmaceutically acceptable salt thereof.

4. A capsule as claimed in claim 2, wherein the proton pump inhibitor is lansoprazole or a pharmaceutically acceptable salt thereof.

5. A capsule as claimed in claim 1, wherein the H2 receptor antagonist is cimetidine, ranitidine, nizatidine, lafutidine, ebrotidine or famotidine, or a diastereoisomer or an enantiomer thereof, or a pharmaceutically acceptable salt of any of the foregoing.

6. A capsule as claimed in claim 5, wherein the H2 receptor antagonist is famotidine or a pharmaceutically acceptable salt thereof.

7. A capsule as claimed in claim 1, wherein the disintegrant is cross-linked polyvinylpyrrolidone, cross-linked sodium carboxymethylcellulose; sodium starch glycolate or low substituted hydroxypropyl cellulose.

8. A capsule as claimed in claim 7, wherein the filler comprises isomalt and/or microcrystalline cellulose.

9. A capsule as claimed in claim 1, wherein, in the PPI-containing component (a), PPI/salt thereof is presented together with the enteric substance as multiple units comprising individual cores of PPI/salt thereof.

10. A capsule as claimed in claim 9, wherein the enteric substance is presented as a discrete coating on the exterior of the PPI units.

11. A process for the preparation of a capsule as defined in any one of the preceding claims, which process comprises:
(I) dry granulating the H2 receptor antagonist along with the disintegrant and the filler; and then
(II) incorporating the granules along with the PPI-containing component (a) in a capsule.

12. A capsule as defined in claim 1 for use in the treatment of a disorder associated with gastric acid secretion.

13. (canceled)

14. A method of treatment of a disorder associated with gastric acid secretion, which method comprises administering a capsule as defined in claim 1 to a patient in need of such treatment.

15. (canceled)
